(19) United States
(12) Patent Application Publication
(10) Pub. No.: US 2020/0131472 A1
(43) Pub. Date: Apr. 30, 2020

(54) CONSTRUCTS AND METHODS FOR ENGINEERING COMPLEX CELL SYSTEMS

(71) Applicant: McMaster University, Hamilton (CA)

(72) Inventors: Ponnambalam Selvaganapathy, Dundas (CA); Alireza Shahin-Shamsabadi, Hamilton (CA)

(73) Assignee: McMaster University, Hamilton (CA)

(21) Appl. No.: 16/667,348

(22) Filed: Oct. 29, 2019

Related U.S. Application Data
(60) Provisional application No. 62/752,145, filed on Oct. 29, 2018.

(51) Int. Cl. C12N 5/00

(52) U.S. Cl. CPC C12N 5/0068; C12N 2513/00; C12N 2535/10; C12N 2533/90; C12N 2539/00; C12N 2533/54; C12N 2537/10

(57) ABSTRACT

This application provides constructs for use as complex cell systems of a desired shape and methods of preparing thereof. The constructs comprise cells contained within a biocompatible gel matrix deposited on a scaffold material optionally cut into defined patterns and impregnated with a crosslinking agent, wherein the biocompatible gel matrix crosslinks upon contact with the crosslinking agent on the scaffold. By stacking multiple alternating layers of the scaffold material cut into defined patterns and the biocompatible gel matrix deposited on the scaffold in defined patterns, complex 3D structures with features like embedded channels are be formed. These complex cell system constructs can be used as in vitro models of biological processes.

### CROSS-REFERENCE TO RELATED APPLICATIONS

The present application claims the benefit of priority from co-pending U.S. provisional patent application No. 62/752,145 filed on Oct. 29, 2018, the contents of which are incorporated herein by reference in their entirety.

### FIELD

The present application relates to constructs of biological material, and in particular, to complex cell systems, and methods for preparing and use thereof.

### BACKGROUND

Although the ultimate goal of tissue engineering is to create functional tissues and organs using cells and natural scaffolds, considerable effort has been directed towards the development of tissue mimics that are not meant to be implanted inside the body but rather used in vitro to facilitate understanding human physiology or disease processes for applications such as basic or diagnostic research (Griffith and Swartz, 2006; Caddeo et al., 2017). The ability to model normal or diseased tissues made of human cells and expose them to different conditions like drug candidates has the potential to make medical investigations like drug discovery faster and more efficient (Nelson et al., 2008). Although flat, two-dimensional (2D) culture systems are dominant and have been used for over a century, growing evidence suggests that under some conditions their results do not represent in vivo response accurately (Duval et al., 2017). This is mostly because of the dimensional limitations of these systems and lack of environmental cues that cells receive in physiological environment (Baker and Chen, 2012). For example, without a nutrient and oxygen gradient in these systems and cells cannot grow on top of each other which changes their morphology and inter-cellular contact (Antoni et al, 2015). As a result of these differences, when 2D systems are used as models of drug development, a significant failure rate is reported (Hutison and Kirk, 2011).

Recently, three-dimensional (3D) systems have been developed that can recapitulate some of the cellular and compositional distribution of extracellular matrix (ECM) in 3D and therefore yield more accurate responses akin to natural tissues (Nelson et al., 2008). Compared to 2D cell culture systems, these 3D models can maintain the phenotype of cells (Abbott, 2003) by controlling the polarity of cell-cell and cell-matrix contacts (Wodarz and Nathke, 2007; Morrison and Kimble, 2006), as well as mechanical properties of their environment (Legant et al, 2010; Huang and Ingber, 1999) and transport characteristics of important soluble growth factors (Griffith and Swartz, 2006). Despite these improvements, many of the methods used to fabricate the 3D models are time consuming, require specialized instruments and are still incapable of completely mimicking natural conditions accurately (Derda et al, 2009). Some of these techniques also have limitations in accurately recreating complex 3D geometries and compositions, scaling up, ability to handle samples after printing, or to extract cells from them for further analysis post culture (Antoni et al, 2015).

For a 3D model to be accurate, it should exhibit the architectural complexities of native tissue. Bioprinting techniques, including inkjet printing, extrusion, layer by layer lamination, or stereolithography, have emerged as a promising approach (Ozbolat et al., 2016; Peng et al., 2016) for this purpose. However, despite their potential, bioprinting techniques still suffer from limitations including, inability to print thicker structures, as in the case of ink-jet printing (Dababneh and Oxbolat, 2014; Kolesky et al., 2016), or are limited to single bio-ink such as in stereolithography (Dababneh and Oxbolat, 2014; Liu et al., 2017). Moreover, the lack of structural strength of many natural ECMs make construction of 3D structures solely from them difficult and often a structural scaffolding material is added. Even with structural scaffolds, current methods still do not have the ability to incorporate multiple ECMs or cell types with predefined patterns nor does it allow integration of perfusion channels that are critical in maintaining appropriate conditions for cell growth in the bulk (Derda et al, 2009; Derda et al, 2011).

### SUMMARY

The present application is directed toward overcoming one or more of the issues discussed above, by using an approach that combines xurography of a structural scaffold material with biomaterial printing for precise distribution of ECMs and cells on it to create complex patterns of biological materials that are difficult or expensive to create otherwise, using simple tools. When followed by assembly in a layer by layer fashion this method is used to create complex 3D cell culture systems that could mimic natural tissue compositions.

Briefly, printing of hydrogel on a cellulose-based paper scaffold pretreated with its crosslinker allows for in situ crosslinking to create any designed pattern of cell and ECM loading on a single layer. Xurographic cutting of the scaffold is used to create perfusion channels in these layers, while layer by layer lamination is used to extend the geometry to 3D. Combining this approach with different cell types, hydrogels as ECMs, and xurographic patterns, the complex geometries and perfusion networks of natural tissues can be recapitulated in vitro with proper 3D distribution of cells, ECMs, and different molecules while providing the whole structure with mechanical stability, direct and easy access to the cells, even the ones that are positioned deep in to the bulk of the structure, without the need to fix or section the samples. These complex 3D structures of multicellular environments with realistic architectural features, such as integrated perfusion networks, mimic natural tissues that model biological processes to enable sophisticated in vitro assays for applications including disease studies, diagnostics research and drug discovery.

Accordingly, the present application provides a cell construct for use as a complex cell system of a desired shape comprising cells within a biocompatible gel matrix deposited in defined patterns on a scaffold material impregnated with a crosslinking agent and pre-cut into a defined pattern prior to deposition of the gel matrix, wherein depositing the gel onto the scaffold results in crosslinking of the gel matrix. In an embodiment, the biocompatible gel matrix is a hydrogel, such as alginate, collagen, or a combination thereof. In an embodiment, the scaffold is comprised of paper or paper-based material comprised of cellulose. In an embodiment, the construct is used as an in vitro model of multicellular environments to study biological processes including, but not limited to, cell migration, proliferation and signaling in response to differences in the extracellular environment.

Also provided is a 3D structure comprised of multiple alternating layers of the gel matrix and scaffold, which have been aligned and stacked. In an embodiment, the defined patterns cut into the scaffold material form voids of both scaffold and gel matrix within the multilayer 3D structure. In a further embodiment, the voids are shaped as perfusion channels embedded in the structure.

In one embodiment, the application provides a construct comprising:

at least one first layer comprising a scaffold, the scaffold comprising a crosslinking agent; and

at least one second layer comprising a biocompatible gel, the biocompatible gel comprising a plurality of cells, wherein the biocompatible gel is crosslinked to the crosslinking agent, and wherein the scaffold comprises at least one pattern cut into the scaffold.

In another embodiment, the pattern defines at least one void.

In another embodiment, the biocompatible gel is present uniformly or in a pattern on the second layer.

In a further embodiment, the construct is a three-dimensional structure comprised of a plurality of alternating layers of the first layer and the second layer, wherein the layers are joined by crosslinking of the biocompatible gel with the crosslinking agent of the adjacent scaffold layer.

In another embodiment, the three-dimensional structure comprises at least one three-dimensional void, wherein the three-dimensional void is defined by at least one pattern cut into at least one scaffold.

In another embodiment, the three-dimensional void is defined by a plurality of patterns cut into a plurality of scaffolds. Optionally, the three-dimensional void comprises or is a channel, an inlet or an outlet.

In another embodiment, the biocompatible gel comprises a hydrogel, optionally wherein the hydrogel is selected from the group consisting of alginate, collagen, gelatin, fibrinogen, chitosan, hyaluronan acid, poly-ethylene glycol, lactic acid, N-isopropyl acrylamide and combinations thereof.

In another embodiment, the scaffold comprises paper or a paper-based material. In a further embodiment, the scaffold comprises a polymer, biodegradable polymer, glass fiber, cellulose, collagen, fibrin, laminin or a combination thereof.

In another embodiment, the plurality of cells comprises mammalian cells, optionally cells selected from the group consisting of hepatocytes, pancreatic Islet cells, fibroblasts, chondrocytes, osteoblasts, endothelial cells, exocrine cells, smooth or skeletal muscle cells, myocytes, adipocytes, ectodermal cells, ductile cells, kidney cells, intestinal cells, parathyroid and thyroid cells, nerve cells, ocular cells, integumentary cells, pluripotent cells and stem cells, and combinations thereof.

The present application also provides a method for preparing a complex cell system construct of a desired shape comprising applying a crosslinking agent on a scaffold material, cutting the scaffold into defined patterns and depositing a layer of biocompatible gel matrix onto the pre-cut scaffold, wherein the biocompatible gel matrix crosslinks upon contact with the crosslinking agent on the scaffold. In an embodiment, cells are embedded in the layer of biocompatible gel matrix prior to deposition onto the scaffold. In an embodiment, deposition of the biocompatible gel matrix is performed by printing. In a further embodiment, the printing technique is extrusion-based. In an embodiment, the scaffold is cut by xurography.

In an embodiment, aligning and stacking a plurality of alternating layers of the scaffold and gel matrix results in a 3D structure joined together by the crosslinking of the gel matrix upon contact with crosslinking agent applied to the adjacent scaffold layer. In an embodiment, cutting at least one scaffold layer into defined patterns creates voids in both the at least one scaffold layer and its adjoining gel matrix layer within the three-dimensional structure. In a further embodiment, the voids are perfusion channels that help retain viability of the cells contained within the 3D structure.

In one embodiment, the application provides a method of preparing a construct comprising:

a) applying a crosslinking agent to a scaffold, and

b) applying a biocompatible gel comprising a plurality of cells onto the scaffold, and

c) cutting at least one pattern into the scaffold before or after the applying the crosslinking agent;

wherein applying the biocompatible gel results in crosslinking of the biocompatible gel with the crosslinking agent.

In one embodiment, the cutting is performed using a technique selected from the group consisting of xurography, laser cutting, laser engraving, etching, or a combination thereof.

In another embodiment, the biocompatible gel is applied to the scaffold uniformly or in a pattern.

In another embodiment, the biocompatible gel layer is applied by printing, optionally wherein the printing is selected from the group consisting of inkjet printing, extrusion, microcontact printing, stereolithography, or a combination thereof.

In another embodiment, the method further comprises aligning and stacking a plurality of alternating layers of the scaffold and the biocompatible gel to provide a 3-dimensional structure, wherein the layers are joined together by crosslinking of the biocompatible gel with the crosslinking agent of the adjacent scaffold.

In a further embodiment, the plurality of scaffolds are aligned and stacked to provide a three-dimensional void in the construct. Optionally, the three-dimensional void comprises or is a channel, an inlet or an outlet.

In another embodiment, the plurality of cells comprises mammalian cells, optionally cells selected from the group consisting of hepatocytes, pancreatic Islet cells, fibroblasts, chondrocytes, osteoblasts, endothelial cells, exocrine cells, smooth or skeletal muscle cells, myocytes, adipocytes, ectodermal cells, ductile cells, kidney cells, intestinal cells, parathyroid and thyroid cells, nerve cells, ocular cells, integumentary cells, pluripotent cells and stem cells, and combinations thereof.

In another embodiment, the method further comprises applying media to the construct and culturing the plurality of cells.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DRAWINGS

The embodiments of the application will now be described in greater detail with reference to the attached drawings in which:

FIG. 1 shows exemplary embodiments of the preparation of constructs of the application where a) printing calcium solution on paper that allows its uniform distribution (lines in the path are 5 mm apart); b) evaluation of printing resolution by printing 10 mm apart hydrogel lines and measuring thickness in different locations; c) printing thin layers of hydrogel with different types of cells embedded in them with high resolution for applications such as cell migration assays; and d) fabrication of 3D structures by cutting the paper, printing different hydrogels with different cells surrounding cut area, and stacking layers, mimicking blood vessel-like structures that are perfusable.

FIG. 2 shows the a) amount of Ca deposited on paper printed with various solution concentrations and print speeds and measured using EDS; b, c) linewidth of the printed gel line with various amount of Ca preloaded on paper; d) effect of flow and feed rate on printed patterns with exemplary optimal conditions producing uniform line printing.

FIG. 3 shows the a) measurement from Alamar blue viability assay; b) intensity of fluorescent light from stained live cells; c) intensity of fluorescent light from stained dead cells; and live/dead images of d) very Low (vL) and e) High (H) Ca samples.

FIG. 4 shows a) schematic of an exemplary checkered pattern printing of HUVECs and 3T3 cells printed using exemplary optimal printing condition on H Ca paper (2 wt % Alg in DMEM, flow rate of 0.5 mL/min and feed rate of 1000 mm/min); b) images of exemplary printed hydrogel checkered pattern; c) a close up image showing sharply defined borders between regions containing different cells in exemplary constructs of the application.

FIG. 5 shows a) images of 3T3s migration in collagen type I in an embodiment of the application; b) effect of FBS content on outwards migration of the cells embedded in collagen in an embodiment of the application; c) effect of FBS content on inwards migration of the cells embedded in collagen in an embodiment of the application; d) cells becoming unviable instead of migrating when the ring is printed deep in alginate in an embodiment of the application; e) cells escaping the environment instead of migrating when the ring is printed close to the top in an embodiment of the application.

FIG. 6 shows a a) schematic of an exemplary fabrication and assembly process to create multicellular patterned 3D structures with perfusion channels integrated; b) top view of printed samples; c) cross-section of printed samples.

FIG. 7 shows the a) change in fluorescent activity of exemplary samples at the center; b) number of clusters in each exemplary condition after 12 days; c, d) low magnification image of exemplary printed samples; e) live/dead stained exemplary samples at day 12.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art. In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this application, the singular forms “a”, “an” and “the” include plural references unless the content clearly dictates otherwise.

In embodiments comprising an “additional” or “second” component (for example, an “additional” or “second” layer), the second component as used herein is different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

### II. Constructs

The present inventors have shown that printing of a cell-containing hydrogel on a cellulose-based paper scaffold pretreated with its crosslinker allows for in situ crosslinking to create a pattern of cell and extracellular matrix (ECM) loading on a single layer. Xurographic cutting of the scaffold can create perfusion channels in these layers, while layer by layer lamination is used to extend the geometry to three dimensions.

Accordingly, the present disclosure provides a construct (for example, a cell or tissue construct) comprising: a first layer comprising a scaffold comprising a crosslinking agent; and a second layer comprising a biocompatible gel matrix comprising a plurality of cells, wherein the biocompatible gel is crosslinked to the crosslinking agent.

As used herein, the term “scaffold” refers to any solid support to which a biocompatible gel can be deposited, printed or applied.

The scaffold is optionally a thin, largely planar material (also referred to as a “sheet”). In one embodiment, the scaffold is about 0.01 mm to about 10 mm thick, optionally about 0.5 mm to about 5 mm, or about 1 mm to about 4 mm thick. In another embodiment, the scaffold has an area of about 0.01 cm² to about 100 cm², optionally about 1 cm² to about 50 cm² or about 5 cm² to about 25 cm². Various scaffold shapes are contemplated herein. In one embodiment, the scaffold is a square, rectangular, circular or oval sheet or a largely square, rectangular, circular or oval sheet.

In one embodiment, the scaffold is a paper or paper-based material. The term “paper” or “paper-based material” as used herein refers to a commodity of thin material produced by the amalgamation of fibers, typically plant fibers composed of cellulose, which are subsequently held together by hydrogen bonding. Optionally, the scaffold comprises or is a polymer, glass fiber, cellulose, collagen, fibrin, gelatin, laminin or a combination thereof. In another embodiment, the scaffold comprises or is a natural extracellular matrix (ECM) that has been be electrospun. Examples of ECM materials include gelatin, collagen and laminin. In another embodiment, the scaffold comprises or is a biodegradable polymer much as polylactic acid (PLA), poly(lactic-co-glycolic acid) (PLGA) or polyvinyl alcohol (PVA).

In one embodiment, the scaffold material, optionally a paper or paper-based material, polymer, biodegradable polymer, glass fiber, cellulose, collagen, fibrin, gelatin, laminin, natural extracellular matrix (ECM) polylactic acid (PLA), poly(lactic-co-glycolic acid) (PLGA) or polyvinyl alcohol (PVA), has been electrospun into membrane form.

In another embodiment, the scaffold is biocompatible and/or biodegradable.

In another embodiment, the scaffold is a semi-transparent or transparent membrane. Transparent membranes can include for example, glass fiber membranes or silicone electrospun membrane or ion track etched membranes made of poly carbonate or polyethylene that have pore sizes below 300 nm.

In one embodiment, the scaffold comprises a crosslinking agent. The term “crosslinking agent” as used herein includes any molecule, atom, or ion that is capable of forming one or more crosslinks between polymer chains. The term “crosslink(s)” or “crosslinking” refers to a comparatively short connecting unit (as in a chemical bond or chemically bonded group), in relation to a monomer, oligomer, or polymer, between neighboring chains of atoms in one or more complex chemical molecule, e.g., a polymers. Suitable cross-linking agents are known in the prior art. For example, when the biocompatible gel is an alginate, the cross-linking agent may be calcium chloride. Different molarities of calcium chloride solutions are contemplated herein, including, but not limited to about 0.1M Ca to about 1M Ca. In another example, when the biocompatible gel is fibrinogen, the crosslinker is optionally thrombin.

The crosslinking agent may cover the entire surface are of the scaffold (i.e, uniform coverage), or it may cover a specific area of the scaffold or be present in a specific pattern on the scaffold. Examples of non-limiting patterns include for example stripes, zig zags, pixelated or ring shaped patterns.

The scaffold is optionally cut before or after the crosslinking agent is applied. As such, the scaffold may include at least one cut. The cut may be made in a pattern. One example of a pattern is a void or multiple voids. As used herein, the term “void” refers to an empty space of gap in the scaffold. The void may be contained completely within the scaffold (for example, a hole entirely within the scaffold) or may be only partially within the scaffold such that not all sides of the void are defined by the scaffold (for example, a notch cut into the edge of the scaffold). As used herein, the term “void” refers to an empty space of gap in the scaffold. The void may be contained completely within the scaffold (for example, a hole entirely within the scaffold) or may be only partially within the scaffold such that not all sides of the void are defined by the scaffold (for example, a notch cut into the edge of the scaffold).

The construct further comprises a second layer comprising a biocompatible gel, wherein the gel is crosslinked to the crosslinking agent. As used herein, the term “biocompatible” refers to material that is not harmful to living tissues. In one embodiment, the biocompatible gel is a hydrogel (a gel in which the liquid component is water). Examples of hydrogels include, but are not limited to, alginate, collagen, gelatin, fibrinogen, chitosan, hyaluronan acid, poly-ethylene glycol, lactic acid, N-isopropyl acrylamide and combinations thereof.

In some embodiments, the biocompatible gel comprises a plurality of cells. In one embodiment, the cells are mammalian cells, optionally cells selected from the group consisting of hepatocytes, pancreatic Islet cells, fibroblasts, chondrocytes, osteoblasts, endothelial cells, exocrine cells, smooth or skeletal muscle cells, myocytes, adipocytes, ectodermal cells, ductile cells, kidney cells, intestinal cells, parathyroid and thyroid cells, nerve cells, ocular cells, integumentary cells, pluripotent cells and stem cells, and combinations thereof. In another embodiment, the cells produce a particular biomaterial of interest, for example a protein, peptide, nucleic acid or lipid. The concentration of cells in the scaffold optionally ranges from about 1×10⁴ cells/ml to about 1×10¹⁰ cells/ml, optionally about 1×10⁵ cells/ml to about 1×10⁷ cells/ml or about 1×10⁶ cells/ml.

The biocompatible gel may comprise a single cell type or a mixture of cell types. Further, when the construct comprises multiple layers, each layer of the biocompatible gel may contain the same cell type or mixture of cell types or different layers may contain different cell types or mixtures of cell types.

The biocompatible gel optionally also includes natural ECM components including, but not limited to, collagen, fibrin, gelatin, laminin and/or matrigel.

The biocompatible gel is applied to the scaffold comprising the crosslinking agent. The biocompatible gel may be applied in a manner known in the art, including, but not limited to, printing such as inkjet printing, extrusion based printing, microcontact printing, stereolithography, or a combination thereof. The biocompatible gel is may be applied such that it covers the entire surface area of the scaffold (for example, in a uniform manner), or it may be applied in a pattern. Examples of patterns include for example stripes, zig zags, pixelated or ring shaped patterns. The biocompatible gel may be applied in the same pattern as the crosslinking agent or in a different pattern.

The construct optionally comprises a plurality of alternating layers of the scaffold and the biocompatible gel, wherein the layers are joined together by crosslinking of the biocompatible gel with the crosslinking agent of the adjacent scaffold. In one embodiment, the biocompatible gel penetrates the adjacent layer and then crosslinks. The plurality of alternating layers thereby creates a 3-dimensional scaffold. The construct optionally contains between 2-100 layers, optionally 4-20 layers.

In one embodiment, the 3D scaffold comprises at least one three-dimensional void, wherein the three-dimensional void is defined by a pattern or patterns cut in the scaffold layers. A person of skill in the art will appreciate that stacking multiple scaffold layers such that voids in each layer are aligned, can result in a 3-dimensional void. As used herein, the term “three-dimensional void” refers to a 3D empty space or gap in the construct. The void may be contained completely within the construct (for example, a 3D hole entirely within the scaffold) or may be only partially within the scaffold such that not all sides of the void are defined by the construct (for example, a notch cut into the edge of the construct). The 3D void is optionally a channel, an inlet or an outlet.

As used herein, the term “channel” refers to a void (optionally a 3D void) that is longer than it is wide. For example, the channel may be about 10 um to about 10 mm in depth and about 100 um to about 1 cm wide. A channel may function for example as a perfusion channel for flowing media/and or nutrients into the construct.

### III. Methods

The present application also provides a method of preparing a cell construct. In one embodiment, the method comprises a) applying a crosslinking agent to a scaffold, and b) depositing a biocompatible gel comprising a plurality of cells onto the scaffold, wherein depositing the biocompatible gel results in crosslinking of the biocompatible gel with the crosslinking.

The crosslinking agent may be applied to the scaffold by any method known in the art. Methods of applying a crosslinking agent to a scaffold include, but are not limited to, printing, dip coating and soaking. The crosslinking agent may be applied such that it covers the entire surface area of the scaffold, or it may be applied to a specific area of the scaffold or in a specific pattern. Examples of patterns include for example, stripes, zig zags, pixelated or ring shaped patterns.

The term “printing” as used herein refers to the placement of a substance, such as a crosslinking agent or biocompatible gel, on the scaffold using a mechanical device that prints the substance onto the scaffold using techniques including, but not limited to, inkjet printing, extrusion, microcontact printing, or stereolithography.

The scaffold is optionally cut into a pattern before or after applying the crosslinking agent. As used herein, the expression “cut into a pattern” means that at least one cut is made to the scaffold. In one embodiment, the cut or pattern is made through the entire thickness of the scaffold. The cut may be made in a pattern. One example of a pattern is a void or multiple voids. As used herein, the term “void” refers to an empty space or gap in the scaffold. The void may be contained completely within the scaffold (for example, a hole entirely within the scaffold) or may be only partially within the scaffold such that not all sides of the void are defined by the scaffold (for example, a notch cut into the edge of the scaffold).

Cutting the scaffold may be performed by any technique known in art. In one embodiment, the scaffold is cut using a subtractive process. In another embodiment, cutting the scaffold is performed by a technique such as xurography, laser cutting, etching, or a combination thereof.

The term “xurography” (or “razor writing”) as used herein refers to a rapid prototyping technique that uses a cutting plotter to cut microstructures in various film materials and does not require a clean room or caustic chemicals as compared to other prototyping techniques.

A biocompatible gel comprising a plurality of cells is applied to the scaffold upon which the cross-linking agent has been applied. As described above, the scaffold is optionally cut prior to the application of the biocompatible gel. In another embodiment, the scaffold is cut after the biocompatible gel is applied.

The biocompatible gel may be applied on the scaffold by any means known in the art. For example, the biocompatible gel is optionally applied by printing, for example, inkjet printing, extrusion, microcontact printing, stereolithography, or a combination thereof.

The biocompatible gel may be applied such that it covers the entire surface area of the scaffold (i.e., a uniform cover), or it may be applied to a specific area of the scaffold or in a specific pattern. Examples of patterns include for example stripes, zig zags, or a pixelated or ring shaped pattern. The pattern by which the biocompatible gel is applied may be the same or different from the pattern by which the crosslinking agent is applied.

Biocompatible gels comprising different cell types or mixtures of cell types may be applied to a single scaffold. In one embodiment, different cells are applied to different locations. For example, vascular cells could be applied close to cut channels and other cell types (for example, muscle cells and fibroblasts) could be applied in the interior away from the channels.

Upon application to the scaffold, the biocompatible gel starts crosslinking immediately to the scaffold that has its crosslinker on it. Accordingly, unintended spread of the biocompatible gel is minimized.

In one embodiment, the method further comprises aligning and stacking a plurality of alternating layers of the scaffold and the biocompatible gel to provide a 3-dimensional structure, wherein the layers are joined together by crosslinking of the biocompatible gel with the crosslinking agent of the adjacent scaffold.

Depending on the pattern or patterns cut into the scaffolds, the 3-dimensional structure may include one or more 3D voids. A person of skill in the art will appreciate that stacking multiple scaffold layers, such the voids in each layer are aligned, can result in a 3-dimensional void. As used herein, the term “three-dimensional void” refers to a 3D empty space or gap in the construct. The void may be contained completely within the construct (for example, a 3D hole entirely within the scaffold) or may be only partially within the scaffold such that not all sides of the void are defined by the construct (for example, a notch cut into the edge of the construct). The 3D void is optionally a channel, an inlet or an outlet. The voids can be channels, which may act as perfusion channels, inlets or outlets.

In one embodiment, the method further comprises culturing the plurality of cells by incubating the construct with an appropriate growth media. In one embodiment, the construct is placed in a container such as a petri dish and immersed in a growth media. In another embodiment, media is added to the channels of the construct.

The constructs described herein can be used for drug screening. Accordingly, also provided herein is a method for screening for activity of a compound of interest comprising treating a construct as described herein with a compound of interest and observing the effect of the compound on the plurality of cells. For example, a compound of interest may be screened for its effect on the growth rate of the cells, the viability of the cells and/or protein expression in the cells. In one embodiment, different doses of the compound of interest may be studied. The compound of interest is optionally a drug candidate, including for example, a small molecule or a biologics.

The constructs described herein can also be used as in vivo or in vitro bioreactors where cells producing specific biomaterials for example, a protein (for example, an antibody), peptide, hormone (for example, insulin), nucleic acid or lipid are included in the biocompatible gel. Accordingly, in such an embodiment, the methods described herein further comprise culturing the plurality of cells and isolating a biomaterial of interest.

The constructs described herein can be further used in regenerative medicine. Accordingly, in such an embodiment, the methods described herein further comprise administering the construct to a subject in need thereof.

Also provided herein are methods of using the constructs of the disclosure as in vitro experimental models.

### EXAMPLES

The following non-limiting examples are illustrative of the present application:

### Example 1. Fabrication Process and Design

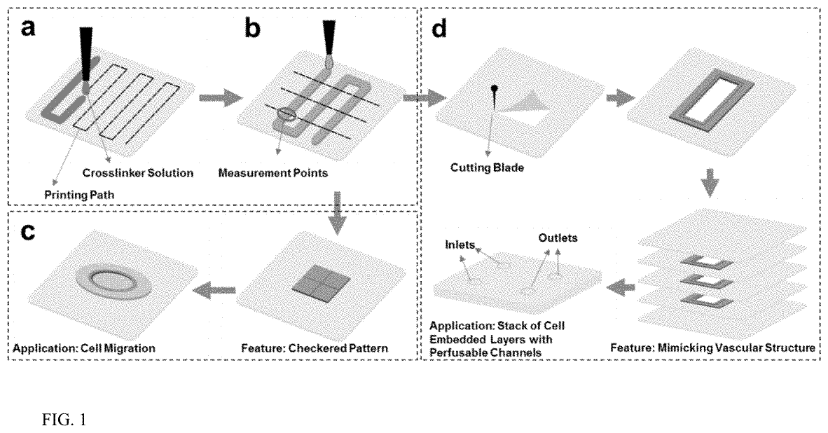

The extrusion printing on cellulose scaffolds with lamination process (shown in FIG. 1) starts by pretreating the paper (Whatman® cellulose chromatography papers, Grade 1 Chr) with the hydrogel's crosslinker (in this case calcium (Ca) as the crosslinker of the sodium alginate) (FIG. 1a). In order to create 2D patterned structures, hydrogel precursors (such as alginate) are then printed (FIG. 1b) which leads to rapid crosslinking once the precursor wicks into the crosslinker soaked paper. Using an extrusion printer various spatial patterns such as a pixelated one or a ring shaped one as shown in FIG. 1c with different type of cells embedded in them is printed. In order to create 3D structures, the pretreated cellulose sheets are cut using automated cutters in the desired patterns that constitute the perfusion channels, inlets, outlets and other features in the 3D structure. Next, hydrogels with different type of cells embedded in them are printed on paper in predefined patterns that reconstitute the biological complexity of a single layer of the final desired 3D structure. Finally, the individually printed layers are stacked one on top of another and allowed to bond with each other to create the final 3D structure. By cutting the paper with different patterns and stacking them properly, perfusable channels will form that provide the proper nutrient and oxygen distribution. Since the hydrogel is crosslinked immediately upon contacting the paper that has its crosslinker on it and does not spread uncontrollably, the need for other steps such as wax printing to confine or pattern the hydrogel placement is eliminated. By printing cells in different patterns and then stacking layers containing different patterns with proper aligning, 3D structures with higher thicknesses proper support of cellular viability can be formed and used as an in vitro model. These layers can also be destacked to have direct access to live cells on each layer. Hydrogels printed on one layer of the paper could also allow easier handling of thin layers of hydrogel that are not mechanically stable.

The first step in the extrusion printing process on a single layer is the uniform printing of the cross-linking agent. This step is done to ensure that there are uniform amounts of cross-linking agent absorbed on the paper scaffold. The absorbed cross-linking agent was then used to instantaneously cross-link any subsequent gel precursor that is printed on top of it and prevent it from spreading within the paper scaffold. In the case of printing alginate gels, calcium chloride is used as the crosslinking agent. Although methods such as dip coating and soaking could be used, it was found that extrusion printing of the cross-linking agent provided a robust method to uniformly deliver defined quantities of crosslinking agent across the entire paper scaffold. Using different molarities of Ca solutions (0.1 and 1 M) and different feedrates (2000, 3000, and 4000 mm/min) 4 different amounts of Ca was deposited on the paper.

In the next step, alginate hydrogel was printed on paper which started crosslinking immediately upon contact due to the presence of the crosslinker on paper. This instantaneous crosslinking prevented excessive spreading of the gel on the hydrophilic scaffold allowing high resolution of printing, which is optimal for fabricating patterns with high complexity. When there was no crosslinker on the paper, the hydrogel spreads quickly in either direction from the printed line due to the capillary forces from the paper matrix and a much broader feature size with diffuse edges was obtained.

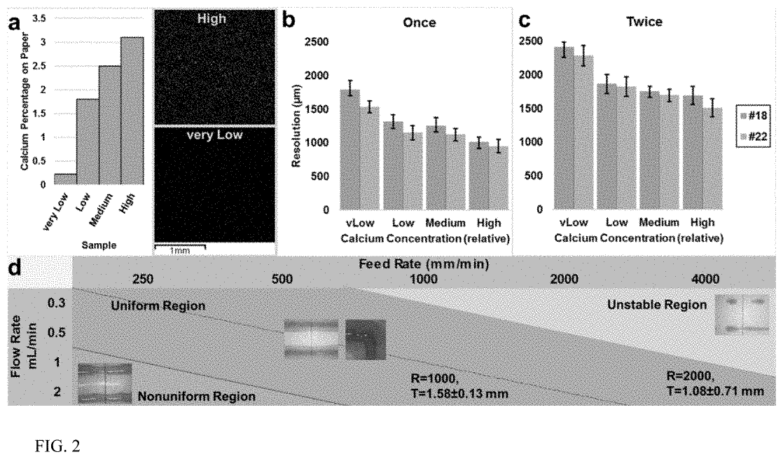

The amount of crosslinker on paper and its distribution could affect the resolution of printing and the viability of the cells. Therefore, paper scaffolds were printed with Ca inks at four different concentrations in a back and forth pattern as shown in FIG. 1a. Elemental analysis on the printed paper scaffold was performed using EDS which showed that the Ca distribution was uniform in all cases and the amount of Ca immobilized on the paper increased with increasing concentration in the ink (FIG. 2a). Next, extrusion printing the alginate on top of the Ca loaded paper scaffold produced a good definition of the defined zig zag pattern as shown in FIGS. 2b and c. The width of the crosslinked gel line that was formed was found to be significantly dependent on the number of a times a line is printed with an increase in line width of −40% between once and twice printed line. When another line of gel is printed on top of previously printed one, it is not in immediate contact the Ca loaded paper scaffold and won't be exposed to as much Ca as the first line. Therefore, it spreads more until it reaches a region of enough Ca concentration to crosslink and immobilize. Surprisingly, the effect of nozzle size was not found to be significant as extrusion from 18 gauge (838 μm inner diameter) and 22 gauge (603 μm inner diameter) produced similar line thicknesses indicating that the capillary process in the paper substrate dominates over the extrusion process in determining the dimensions of the printing. In summary, printing resolution is lower in lower Ca content or when two layers are printed on top of each other but the effect of nozzle size (18 gauge versus 22 gauge) is not meaningful.

The effect of flow rate and the feed rate of the extrusion printing process on the dimensions of the printed line was also determined. Alginate (2 wt % solution) was printed on top of H Ca (high calcium content) paper at various feed rates between 250 and 4000 mm/min and flow rates between 0.25 and 2 mL/min with 18 gauge needle. Interestingly, when feed rate to flow rate ratio (R) was between 1000 and 2000 mm/mL, a uniform line was printed on the paper with a linewidth of 1.08±0.07 mm and 1.59±0.13 mm for ratios of 2000 and 1000 mm/mL respectively, as shown in FIG. 2d. At higher ratios, the linewidth is non-uniform while at lower ratios the printing was intermittent. Such optimal ratios are often seen in extrusion of non-Newtonian fluids where the flow rate and feed rate are well matched to produce printed lines with well-defined and uniform edges. Alginate lines were also printed with the printhead moving in different directions (top to bottom and bottom to top) to determine if the direction of printing has any effect on the thickness and uniformity of the printed line (FIG. 1b). Images at three different locations along each line (top, middle, and bottom) was measured. In all the samples printed within the “stable” region, width of the line was measured at various locations along the printed zig zag pattern and it was found to be nearly the same. Although the printed lines demonstrated here are ˜1 mm in width, thinner lines could be printed by extruding through smaller nozzles and onto papers that are thinner.

### Example 2. Viability Assessment

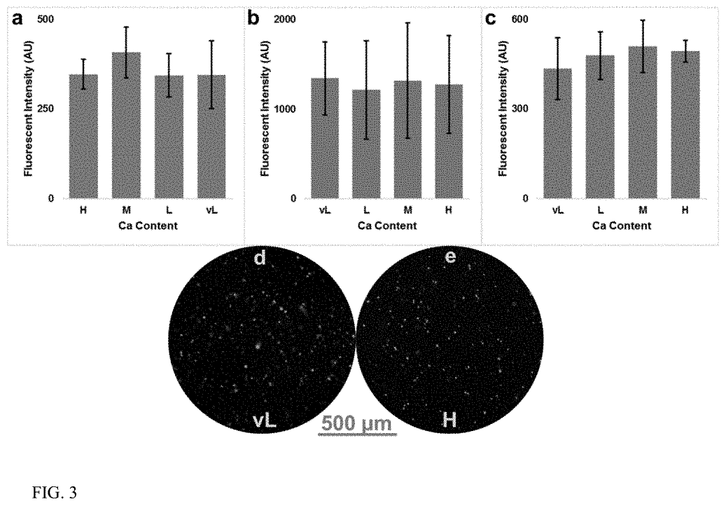

After determining the conditions for stable printing of alginate gels, a bioink was prepared by mixing 2 wt % alginate with 3T3 mouse fibroblasts with the concentration of 1×10⁶ cells/mL and used for printing. To study effect of printing process and Ca concentration on paper on the cells' viability, two different assays were performed on printed lines that were fabricated with the optimized “stable” printing conditions. Metabolic assays such as MTT ((3-(4,5-dimethylthiazol-2-yl)-2,5-diphenyltetrazolium bromide)) assay or Alamar blue assay (ABA) that are typically used for assessing cell viability in plate based assays are not be suitable in the present case as the printed patterns that were compared had varying amounts of crosslinking due to the different amounts of Ca loaded on the paper substrate. Consequently, the amount of crosslinking and therefore the mass transfer through these gels could potentially be different which could affect the results obtained. Therefore, the traditional ABA assay was modified with an additional step where 0.1 M sodium tricitrate solution in Dulbecco's modified Eagle medium (DMEM), which is known to dissolve Ca crosslinked alginate, was added to the samples after printing so that the matrix is dissolved and the cells precipitate on the surface of the paper. ABA assay was performed on the precipitated cells after a few hours to allow complete cell attachment. To further study the number of live and dead cells in each case, live/dead staining was also performed and the amount of fluorescence corresponding to the live and dead cells was measured using a plate reader. The autofluorescence background of chromatography paper was also measured prior to staining and subtracted from the measurement for normalization. It was clear, as shown in FIG. 3, that there is no meaningful difference between metabolic activity and number of live and dead cells of all the conditions of calcium loading on paper chosen in this study indicating that the conditions of printing were benign and did not affect cell viability. Images of the live/dead stained cells also show acceptable number of live cells in these samples.

### Example 3. 2D or 3D Structures and Cell Migration Application

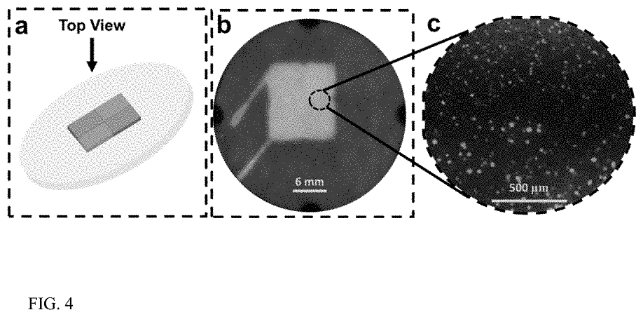

In order to demonstrate the ability of extrusion printing of gels on crosslinker loaded scaffolds, a checkerboard pattern was designed (FIG. 4a) consisting of alternate regions of gels containing two different types of cells. Each of the square regions was designed to be 6×6 mm in size and represent a pixel element. Each pixel element was filled by the parallel hatching style shown in FIG. 4a with a spacing of 0.5 mm. FIG. 4b shows the checkered pattern printed with gfp-3T3 and rfp-HUVEC cells in alternate squares of the checkerboard pattern. The definition of the squares was found to be accurate and the borders between the regions containing alginate loaded with different cells were sharply defined as shown in FIG. 4c without any mixing. By combining various patterns of these pixel elements complex designs could be printed in 2D. Interestingly, since the paper is prepatterned with the crosslinker of the hydrogel, it is possible accurately control the amount of crosslinking spatially as well.

The ability to define sharp boundaries between different hydrogels can be used to perform cell migration studies under different conditions. Cell migration is an important step in different physiological processes such as embryonic development, immune response and healing, as well as in pathological conditions such as cancer metastasis that can be studied using 2D techniques such as wound closure and transwell migration assays (Justus et al., 2014). Although these assays provide valuable information, they are 2D in vitro models (single layer of cells on a plastic substrate) and cannot accurately recapitulate 3D in vivo environment.

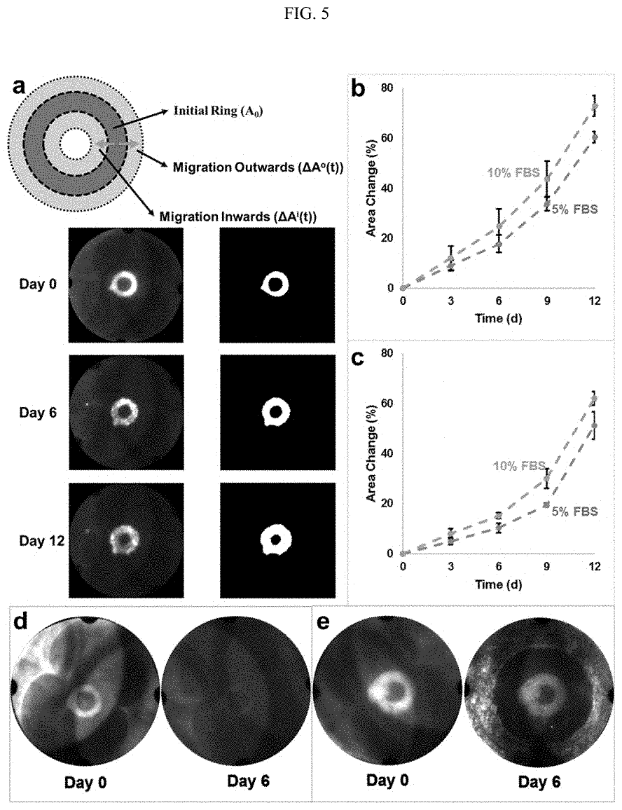

In order to demonstrate the versatility of this process in constructing suitable multimaterial 3D structures for this assay, cells were printed in a ring like structure (cells were embedded in alginate and with its crosslinking, the structure of the ring was preserved). The printed ring was then embedded in either alginate or collagen and their migration was tracked over time by measuring the change in area inside and outside of the initially printed ring. Not only can this technique can be used to study the effect of ECM on cell migration, but by changing the composition in culture media (such as the amount of nutrients or drugs) it could be used to study effect of chemicals in the environment on cell migration as well. FIG. 5a shows the images taken by the ChemiDoc system and analyzed using a Matlab image processing of cells embedded in collagen provided with 5% V/V fetal bovine serum (FBS) supplemented culture media. It also shows the inward and outward migration pattern for cells in collagen with both 5 and 10% V/V FBS (FIG. 5b). As it is shown in FIGS. 5b and c, cells migrate faster when the concentration FBS in the surrounding media is increased which sets up a concentration gradient of this nutrient in the 3D ring that promotes migration. Interestingly, they migrate faster toward the center of the ring rather than outwards. It could be because cells prefer to maintain their cell-cell interaction rather than losing their connection. While cells were able to migrate in a certain pattern in collagen, when they were embedded in alginate they were unable to migrate (FIGS. 5d and 5e). Various scenarios were then investigated for alginate embedded cells by printing a ring of alginate loaded with cells where some of the cells are in close proximity to the culture media and contrasting its behavior to when another layer of cell free alginate was present on the top. In the case where the cells in the 3D printed ring was printed close to the surrounding media, the cells escaped from the alginate gel after a few days. However, when the 3D ring was embedded underneath another layer of crosslinked alginate, the cells quickly became unviable and the fluorescent intensity decreased dramatically. It could have been because of the lack of ligands like arginine-glycine-aspartic acid (RGD) on alginate that cells could attach to and migrate (Ho et al., 2017).

### Example 4. Assembly of 3D Scaffolds and Perfusion Channel Model Application

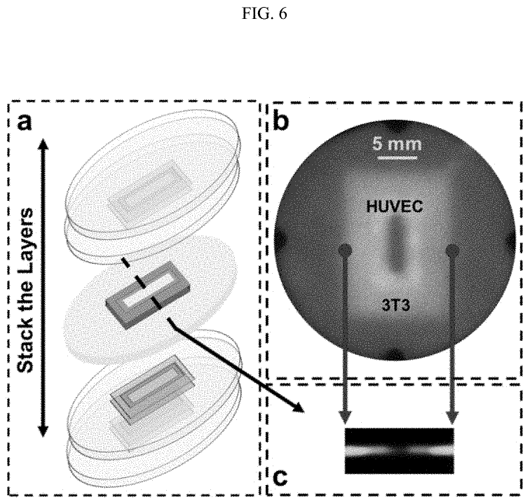

In order to demonstrate the ability of this approach to create complex multicellular 3D constructs, a perfusable 3D structure was designed that contains hollow channels that could be extrusion printed layer by layer and then assembled with alignment by lamination. The hollow channels could be used to perfuse cell culture media to keep the cells in the bulk of the structure alive. The construct consisted of several layers (as shown in FIG. 6a) some of which were xurographically cut to have empty channel structures in the paper scaffold. The xurographically cut layers consisted of alginate loaded with HUVEC cells that were extrusion printed at the borders close to the cut channel regions. They also had alginate loaded with 3T3s printed elsewhere. Lamination of these layers together led to the formation of a 3D bioink printed and loaded scaffold that had hollow perfusion channels inbuilt. During the lamination process, the top part of the gel on each layer is not completely crosslinked and when another layer of paper is placed on top of it, it comes to direct contact with a new layer of paper with Ca on it that helps it to crosslink faster and bond to the top layer. With the extrusion printing, an inner lining of HUVEC loaded gel layer was created surrounded by fibroblasts as shown in FIGS. 6b and c.

These channels that were formed can be easily perfused without leaking, with two parallel channels created by stacking 4 layers of paper with hydrogel printed on them and also two crossing channels each consisted of 2 layers of paper with hydrogel printed on them.

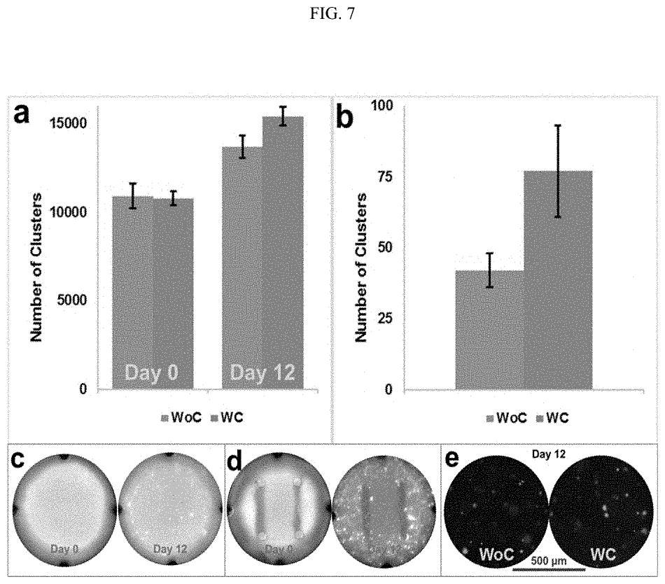

To show the effect of perfusion channels in 3D in vitro models, 3D structures fabricated by stacking three layers of paper with hydrogel printed on them was prepared in which one of them had two parallel 2 mm wide channels that were placed 9 mm apart embedded in it. Using the plate-reader the amount of fluorescent intensity in the center part of each of the samples were measured every 2 days (FIG. 7a). To mimic the perfusion in the channels culture media was pipetted into each of the channels every 12 hrs. As shown in FIG. 7a, after 12 days, fluorescence intensity of cells was higher in the WC samples. The cells form clusters and the number of cell aggregates (FIG. 7b) at day 12 was higher in WC samples and they were located mostly close to the channels (FIGS. 7c and d). The same formation of clusters for other cells in alginate has been previously reported (Guillaume et al., 2015) and has been attributed to lack of cell-adhesivity ligands such as RGD sequence (Koo et al., 2002). Live/Dead stained images of samples (FIG. 7e) also indicate lower live cells and higher dead cells in the center part of WoC samples as opposed to WC channels. Although in both samples cells started to die after a few days but presence of channels, although far from each other, helped retain the viability of cells closer to them as seen from the distribution of clusters. Increasing the density of perfusion channels by reducing the spacing between them is likely to improve the access to nutrients further and ensure cell growth throughout the 3D structure. Therefore, this extrusion printing on cellulose scaffolds with lamination process is versatile in forming hydrogel structures with various cell types embedded in them and integrated with perfusion channels.

Materials and Methods for Examples 1-4

Fabrication Process: For bioprinting purposes, an open source plastic 3D printer (MK2S, PrusaInc) was modified. A holder was designed to replace the printhead with a stainless steel needle as the printing nozzle. The needle was connected using tubing to a syringe attached to a syringe pump that enable extrusion of its contents. The print bed had a vacuum port and appropriate holes that keeps the paper in the stage by applying vacuum from below. To pretreat the chromatography paper (Whatman® 1 Chr, 0.180) with Ca as the crosslinker of the chosen hydrogel (Alginic Acid Sodium Salt-Sigma), two different calcium concentrations (0.1 and 1 M) with different printing speeds (2000, 3000, and 4000 mm/min) were printed on paper in a certain pattern with the flow rate of 0.5 mL/min (FIG. 1 a). After printing, sufficient time was given so that the Ca solution could penetrate to the whole paper and distribute uniformly. The printed paper was kept at room temperature overnight in the sterile environment so that the excess water evaporated, and was cut to desired patterns for further use. To find out the amount of calcium for each condition and to make sure they have uniform distributions, Energy-dispersive X-ray spectroscopy (EDS) analysis was performed in each case in map mode. Based on the initial concentration of the used solution and the speed rates used, 4 different quantified concentrations (called High (H) (1 M and 2000 mm/min), Medium (M) (1 M and 3000 mm/min), Low (L) (1 M and 4000 mm/min), and very Low (vL) (0.1 M and 2000 mm/min)) were prepared for further use.

Different parameters effective on the printing resolution were studied: Ca content of the paper, gauge number of the needle, flow rate of the hydrogel from nozzle, and speed of the nozzle movement (feed rate). In all cases 2 wt % alginate was used. For the characterization purposes, a zigzag pattern was printed and immediately after printing pictures of three different locations of each line (top, middle, and bottom) were taken using a stereo microscope and thickness of the line was measured using ImageJ (FIG. 1b). To study effect of printing layers of hydrogel on top of each other on the resolution, in the next step the same pattern was printed twice (double layer format) and characterization was performed the same as before. The distance between the tip of the nozzle and the paper for the first layer was kept constant at 0.5 mm and for printing more than one layer, it was increase by 0.5 mm for each additional layer. Using these information three different situations were defined, “uniform” condition where the line had uniform thickness (less than 10% variation in thickness), “unstable” condition where the line was not continuous, and “nonuniform” where the line was not steady.

Viability Assessment: Effect of different steps in the process including stress applied to the cells during the process and Ca concentration, on cell viability was studied using designed protocols based on AlamarBlue and live/dead assays (called ABA and LDA, respectively). Briefly, alginate was dissolved in DMEM culture media to reach the concentration of 2 wt %. 3T3 Fibroblast cells were cultured in DMEM supplemented with 10 wt % FBS. After reaching 90% confluence, trypsinized, counted, and gently mixed with alginate solution to have final concentration of 1×10⁶ cells/mL. All 4 type of papers were cut using a Xurographic cutting machine (Silhouette®, Cameo) to the size of 6 well plates (diameter of 34 mm). A circle with diameter of 28 mm was printed with optimal printing condition found in previous section (0.5 mL/min, 1000 mm/min, gauge #18) on the center part of the paper in a double layer format. After printing, each sample was kept on the printing bed for 5 min to complete the crosslinking process, transferred to the wells, washed with phosphate buffered saline (PBS) solution to get rid of extra calcium on the paper, 2 mL of culture media was added to each well, and transferred to an incubator. Viability assays were performed 16 hrs later.

For ABA, culture media was replaced with 2 mL of new culture media supplemented with 0.1 M sodium tricitrate to help dissolve the alginate solution. The samples were transferred to the incubator once again for 2 hours to allow citrate to dissolve the alginate and the cells to attach to the paper. Culture media was changed with normal media to avoid any toxicity caused by excess citrate following by another 4 hours of incubation. Alamar Blue solution was added (10% V/V) to each well and incubated for 1.5 hrs. At the end, 100 μL samples of culture media in each well were transferred to a black 96 well-plate and reading was done at excitation and emission of 560 and 590 nm. Hydrogel samples printed without cells were used as control in each case and the same dissolving process was performed. Finally, difference between fluorescence activity of each case with its control was reported.

For LDA, after 16 hrs of incubation, culture media was aspirated and washed with PBS twice. Because of the inherent auto-fluorescence of the paper, florescent reading was performed before staining using a plate reader at excitations and emissions of 485 and 530 nm for live and 530 and 645 for dead cells as control in multiple regions of the sample. Live/Dead staining of the samples were performed using Calcein/Ethidium Bromide homodimer kit following the instructions. After staining, samples were washed with PBS again and the same reading was performed. Pre-staining amounts were subtracted from post-staining images and reported as final values.

2D Structures—Patterning of Cells and ECMs on Paper: To show the ability of the technique to print complex cellular structures on paper with acceptable accuracy without mixing and contamination of adjacent regions with each other, checkered patterns of cells were printed on paper. Solutions of 2 wt % alginate embedded with green fluorescent protein tagged 3T3s (gfp-3 T3) and red fluorescent protein tagged human umbilical vein endothelial cells (rfp-HUVEC) were printed in a checkered pattern and images were taken using a ChemiDoc™ MP imaging system (Bio-Rad) and an upright microscope. Printing was performed using previously defined optimal conditions. Squares of 6 mm were printed that were 0.5 mm apart from each other (FIG. 1c).

2D Application—Cell Migration Evaluation: The 2D patterning feature of this bioprinting technique was used to study in vitro cell migration in 3D (thick gel layer) environment by printing on only one layer. vL paper was used; hydrogel with 10×10⁶ cells/mL of gfp-3 T3 cells were printed in a ring like structure with diameter of 6 mm. This ring was then embedded in a bigger circular area of alginate without any cells in it that filled both inside and outside area of the ring. The printed samples were transferred to 6 well-plates and culture media was replaced every 3 days. Images of the whole structure were taken every 3 days using ChemiDoc system and analyzed using MatLab image processing toolbox to measure migration of the cells toward inside and outside of the ring. To further study the effect of ECM type on cell migration, the same assay was performed but instead of embedding the ring in a circle of alginate, it was embedded in bovine collagen type I (Life Technologies) with concentration of 4 mg/mL and the same process was performed to measure cell migration. Effect of fetal bovine serum (FBS) content of media on migration speed was also studied by adding 5% V/V and 10% V/V FBS containing DMEM to samples of ring embedded in collagen.

3D Structures—Layer by Layer Assembly of 2D Patterned Scaffolds: In order to demonstrate the ability to construct 3D structures, a perfusion channel with multicellular patterning was designed (FIG. 1d). Channel designs with 2 mm width attached to inlet and outlet regions were cut into the Ca loaded paper layers to serve as the scaffold. Next, hydrogels were printed on paper but not inside the channels and layers of paper were stacked to create a 3D structure in which channels were parallel to each other or crossed each other in different layers. To show these channels were perfusable without leaking, different solutions with different colors were perfused in each of the channels using a syringe pump. Patterned multicellular constructs were created by printing a layer of hydrogel with rfp-HUVEC cells in the regions of the paper scaffold surrounding the channel structure and gfp-3T3 cells in the surrounding regions around to HUVEC cells. By stacking and aligning these bioprinted papers a 3D structure with channels were fabricated that resembled the natural distribution of the cells. To verify proper distribution of hydrogels and cells with distinguishable borders of cells without unintended mixing, images of fluorescent cells were taken using ChemiDoc system. To show the paper was able to support the hydrogel and prevent it from filling the channels, printed and stacked samples were flash-frozen and broken in half and images of the cross-section were taken.

3D Application—Cell Viability in Stacked Layers and Effect of Channels: Importance of perfusable channels in a 3D structure on cell viability was studied by fabricating multilayer stacked structure of cells. Two parallel channels were cut on the paper scaffold. Then, hydrogel with 2×10⁶ cells/mL (gfp-3T3 cells) was printed on the paper and these layers were stacked by proper aligning of the channels (called WC (“with channel”) samples). The same hydrogel patterns were also made on scaffolds without channels (called WoC (“without channel”) samples) and used for comparison. In each case, three layers of paper with channel structures and printed hydrogel on top of them were stacked and finally a last layer of paper, with inlets and outlets for WC samples, added on top to seal the whole structure. Samples were transferred to 6 well-plates and 2 mL culture media was added to each well and changed every two days. Culture media was also pipetted into the channels every 12 hrs in order to replace the media present inside them. To measure the proliferation of cells in each case, fluorescence intensity of the cells was measured every two days using a plate-reader in the center part of each sample and the change in their fluorescent intensity was compared to each other over time. To study effect of having channels on cell distribution, images of the samples were taken using ChemiDoc system and the number of cell clusters were measured using ImageJ. Live/Dead staining was also performed to study the effect of channels on cellular viability. After staining was done, each layer with the hydrogel on top of it was carefully detached by applying a slight shear force and images of the middle layer that had the least access to the surrounding environment were taken using an upright microscope.

### CONCLUSION

In summary, a new bioprinting technique has been developed that is based on printing hydrogel on paper and its in situ crosslinking on paper pretreated with the hydrogel's crosslinker. Paper provides mechanical stability that thin hydrogel layers lack and allows for patterned printing of complex structures out of soft materials. This bioprinting method together with cutting the paper layers and stacking them has the capability to fabricate structures that recapitulate some of the biological complexity of tissues in vitro and can be used as a powerful biological model for drug discovery. In the proof of the concept demonstrated herein, the ability to fabricate checkered patterns on one layer of paper and fabrication of perfusion channels embedded in the bulk of the structure with co-axial structure of endothelial cells and fibroblast was shown. Further optimization of this process like replacing the paper with a completely transparent membrane that allows better microscopy will enable production of in vitro models with a wide range of applications. Furthermore, improving the resolution of cutting and patterning to allow closely spaced channels can allow the entire bulk of the structure to access nutrients sufficiently.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### REFERENCES

1. Griffith, L. G. and M. A. Swartz, Capturing complex 3D tissue physiology in vitro. Nat Rev Mol Cell Biol, 2006. 7(3): p. 211-24.
2. Caddeo, S., M. Boffito, and S. Sartori, Tissue Engineering Approaches in the Design of Healthy and Pathological In Vitro Tissue Models. Front BioengBiotechnol, 2017. 5: p. 40.
3. Nelson, C. M., J. L. Inman, and M. J. Bissell, Three-dimensional lithographically defined organotypic tissue arrays for quantitative analysis of morphogenesis and neoplastic progression. Nat Protoc, 2008. 3(4): p. 674-8.
4. Duval, K., et al., Modeling Physiological Events in 2D vs. 3D Cell Culture. Physiology (Bethesda), 2017. 32(4): p. 266-277.
5. Baker, B. M. and C. S. Chen, Deconstructing the third dimension: how 3D culture microenvironments alter cellular cues. J Cell Sci, 2012. 125(Pt 13): p. 3015-24.
6. Antoni, D., et al., Three-dimensional cell culture: a breakthrough in vivo. Int J MolSci, 2015. 16(3): p. 5517-27.
7. Hutchinson, L. and R. Kirk, High drug attrition rates—where are we going wrong? Nat Rev ClinOncol, 2011. 8(4): p. 189-90.
8. Abbott, A., Cell culture: biology's new dimension. Nature, 2003. 424(6951): p. 870-2.
9. Wodarz, A. and I. Nathke, Cell polarity in development and cancer. Nat Cell Biol, 2007. 9(9): p. 1016-24.
10. Morrison, S. J. and J. Kimble, Asymmetric and symmetric stem-cell divisions in development and cancer. Nature, 2006. 441(7097): p. 1068-74.
11. Legant, W. R., et al., Measurement of mechanical tractions exerted by cells in three-dimensional matrices. Nat Methods, 2010. 7(12): p. 969-71.
12. Huang, S. and D. E. Ingber, The structural and mechanical complexity of cell-growth control. Nat Cell Biol, 1999. 1(5): p. E131-8.
13. Derda, R., et al., Paper-supported 3D cell culture for tissue-based bioassays. Proc Natl AcadSci USA, 2009. 106(44): p. 18457-62.
14. Ozbolat, I. T., W. Peng, and V. Ozbolat, Application areas of 3D bioprinting. Drug Discov Today, 2016. 21(8): p. 1257-71.
15. Peng, W., D. Unutmaz, and I. T. Ozbolat, Bioprinting towards Physiologically Relevant Tissue Models for Pharmaceutics. Trends Biotechnol, 2016. 34(9): p. 722-32.
16. Dababneh, A. B. and I. T. Ozbolat, Bioprinting Technology: A Current State-of-the-Art Review. Journal of Manufacturing Science and Engineering, 2014. 136(6): p. 061016-061016-11.
17. Kolesky, D. B., et al., Three-dimensional bioprinting of thick vascularized tissues. Proc Natl AcadSci USA, 2016. 113(12): p. 3179-84.
18. Liu, W., et al., Rapid Continuous Multimaterial Extrusion Bioprinting. Adv Mater, 2017. 29(3).
19. Derda, R., et al., Multizone paper platform for 3D cell cultures. PLoS One, 2011. 6(5): p. e18940.
20. Justus, C. R., et al., In vitro cell migration and invasion assays. J Vis Exp, 2014(88).
21. Ho, S. S., et al., Cell Migration and Bone Formation from Mesenchymal Stem Cell Spheroids in Alginate Hydrogels Are Regulated by Adhesive Ligand Density. Biomacromolecules, 2017. 18(12): p. 4331-4340.
22. Guillaume, O., et al., Enhancing cell migration in shape-memory alginate-collagen composite scaffolds: In vitro and ex vivo assessment for intervertebral disc repair. J BiomaterAppl, 2015. 29(9): p. 1230-46.
23. Koo, L. Y., et al., Co-regulation of cell adhesion by nanoscale RGD organization and mechanical stimulus. J Cell Sci, 2002. 115(Pt 7): p. 1423-33.

1. A construct comprising:
a) at least one first layer comprising a scaffold, the scaffold comprising a crosslinking agent; and
b) at least one second layer comprising a biocompatible gel, the biocompatible gel comprising a plurality of cells, wherein the biocompatible gel is crosslinked to the crosslinking agent,
and wherein the scaffold comprises at least one pattern cut into the scaffold.

2. The construct of claim 1, wherein the pattern defines at least one void.

3. The construct of claim 1, wherein the biocompatible gel is present in a pattern on the at least one second layer.

4. The construct of claim 1, wherein the construct is a three-dimensional structure comprised of a plurality of alternating layers of the first layer and the second layer, wherein the layers are joined by crosslinking of the biocompatible gel with the crosslinking agent of an adjacent scaffold layer.

5. The construct of claim 4, comprising at least one three-dimensional void, wherein the three-dimensional void is defined by at least one pattern cut into at least one scaffold.

6. The construct of claim 5, wherein the three-dimensional void is defined by a plurality of patterns cut into a plurality of scaffolds.

7. The construct of claim 5, wherein the three-dimensional void comprises or is a channel, an inlet or an outlet.

8. The construct of claim 1, wherein the biocompatible gel comprises a hydrogel, optionally wherein the hydrogel is selected from the group consisting of alginate, collagen, gelatin, fibrinogen, chitosan, hyaluronan acid, poly-ethylene glycol, lactic acid, N-isopropyl acrylamide and combinations thereof.

9. The construct of claim 1, wherein the scaffold comprises paper or a paper-based material.

10. The construct of claim 1, wherein the scaffold comprises a polymer, biodegradable polymer, glass fiber, cellulose, collagen, fibrin, laminin or a combination thereof.

11. The construct of claim 1, wherein the plurality of cells comprises mammalian cells, optionally cells selected from the group consisting of hepatocytes, pancreatic Islet cells, fibroblasts, chondrocytes, osteoblasts, endothelial cells, exocrine cells, smooth or skeletal muscle cells, myocytes, adipocytes, ectodermal cells, ductile cells, kidney cells, intestinal cells, parathyroid and thyroid cells, nerve cells, ocular cells, integumentary cells, pluripotent cells and stem cells, and combinations thereof.

12. A method of preparing a construct comprising:
a) applying a crosslinking agent to a scaffold, and
b) applying a biocompatible gel comprising a plurality of cells onto the scaffold, and
c) cutting at least one pattern into the scaffold before or after the applying the crosslinking agent;
wherein applying the biocompatible gel results in crosslinking of the biocompatible gel with the crosslinking agent.

13. The method of claim 12, wherein the cutting is performed using a technique selected from the group consisting of xurography, laser cutting, laser engraving, etching, or a combination thereof.

14. The method of claim 12, wherein the biocompatible gel is applied onto the scaffold uniformly or in a pattern.

15. The method of claim 12, wherein the biocompatible gel layer is applied by printing, optionally wherein the printing is selected from the group consisting of inkjet printing, extrusion, microcontact printing, stereolithography, or a combination thereof.

16. The method of claim 12, further comprising aligning and stacking a plurality of alternating layers of the scaffold and the biocompatible gel to provide a 3-dimensional structure, wherein the layers are joined together by crosslinking of the biocompatible gel with the crosslinking agent of the adjacent scaffold.

17. The method of claim 16, wherein the plurality of scaffolds are aligned and stacked to provide a three-dimensional void in the construct.

18. The method of claim 17, wherein the three-dimensional void comprises or is a channel, an inlet or an outlet.

19. The method of claim 12, wherein the plurality of cells comprises mammalian cells, optionally cells selected from the group consisting of hepatocytes, pancreatic Islet cells, fibroblasts, chondrocytes, osteoblasts, endothelial cells, exocrine cells, smooth or skeletal muscle cells, myocytes, adipocytes, ectodermal cells, ductile cells, kidney cells, intestinal cells, parathyroid and thyroid cells, nerve cells, ocular cells, integumentary cells, pluripotent cells and stem cells, and combinations thereof.

20. The method of 12, further comprising applying media to the construct and culturing the plurality of cells.
